(19) United States
(12) Patent Application Publication
(10) Pub. No.: US 2006/0004411 A1
(43) Pub. Date: Jan. 5, 2006

(54) Package containing a first housing for storing unused disposable cleaning products and a second housing for storing used cleaning products

(76) Inventor: Aram M. Kadish, Los Angeles, CA (US)

Correspondence Address: CROWELL & MORING LLP;INTELLECTUAL PROPERTY GROUP, P.O. BOX 14300, WASHINGTON, DC, 20044-4300, US

(21) Appl. No.: 11/149,580

(22) Filed: Jun. 9, 2005

Related U.S. Application Data
(60) Provisional application No. 60/583,892, filed on Jun. 30, 2004.

(51) Int. Cl. A61B 17/04

(52) U.S. Cl. 606/233

(57) ABSTRACT

An exemplary package is disclosed including a first housing for storing unused, disposable cleaning products (e.g., tissue papers, paper towels, napkins, moist wipes, etc.), and a second housing for storing used disposable cleaning products, wherein the first and second housing are attached to each other. As an example, a user retrieves unused tissue papers from the first housing, uses the tissue papers, and then disposes the used tissue papers into the second housing.

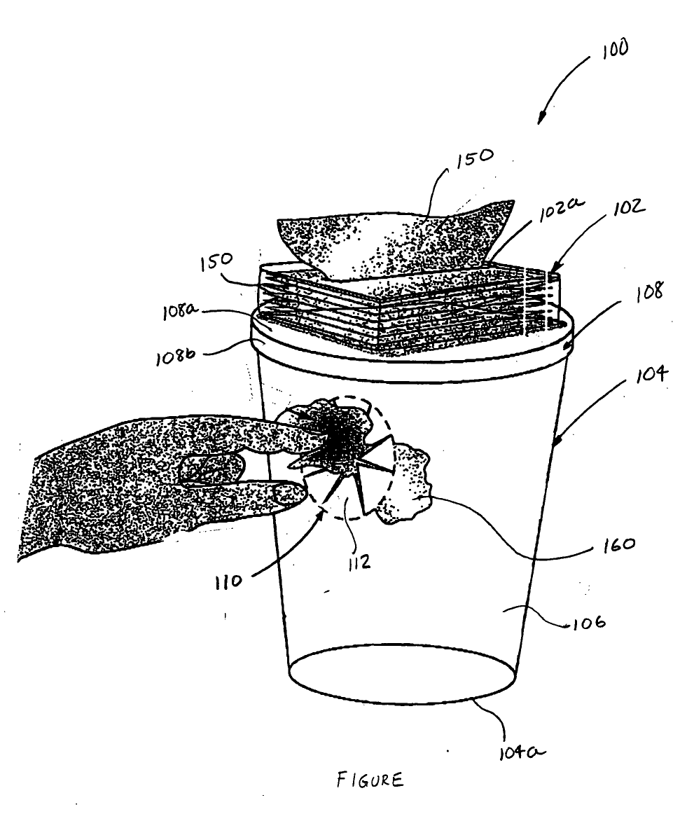

### CROSS-REFERENCE TO RELATED APPLICATIONS

This application claims the benefit of the filing date of Provisional Application, Ser. No. 60/583,892, filed on Jun. 30, 2004, which is incorporated herein by reference. This application is also related to non-provisional application entitled, “Package Containing a First Compartment for Storing Unused Disposable Cleaning Products and a Second Compartment for Storing Used, Disposable Cleaning Products,” filed concurrently with this application.

### FIELD OF THE INVENTION

This invention relates generally to packages, and in particular, to a package containing a first housing for storing unused, disposable cleaning products and a second housing for storing used, disposable cleaning products, wherein the first housing is attached to the second housing.

### BACKGROUND OF THE INVENTION

Disposable cleaning products, such as tissue papers, papers towels, napkins, moist wipes, etc., are usually stored in some type of package. For example, unused tissue papers are typically stored in a cardboard box containing a perforated cover. A user typically tears open the perforated cover to access the unused tissue papers. After using the tissue paper, a user may dispose the tissue paper in a waste basket.

In many situations, there may not be a waste basket or other container available in which to dispose the used tissue paper or other disposable cleaning product. In such situation, a user may place the used tissue paper in an undesirable location, such as on top of a table, on a chair, on the floor, or in one's pocket. Such undesirable disposal of the used tissue paper may lead to non-environmentally friendly disposal and may also lead to unsanitary conditions, which may result in illness in people who come in contact, directly or indirectly, with the used tissue paper.

### SUMMARY OF THE INVENTION

An aspect of the invention relates to an exemplary package for storing both unused, disposable cleaning products and used, disposable cleaning products. The exemplary package comprises a first housing for storing unused, disposable cleaning products, and a second housing for storing used, disposable cleaning products, wherein the first and second housing are attached to each other. A user retrieves unused, disposable cleaning products from the first housing, uses the disposable cleaning products, and then disposes the used, disposable cleaning products into the second housing.

Other aspects, features, and techniques of the invention will be apparent to one skilled in the relevant art in view of the following detailed description of the exemplary embodiments of the invention.

### BRIEF DESCRIPTION OF THE DRAWINGS

FIGURE illustrates a perspective view of an exemplary package in accordance with an embodiment of the invention.

### DETAILED DESCRIPTION OF THE EXEMPLARY EMBODIMENT

FIGURE illustrates a perspective view of an exemplary package 100 in accordance with an embodiment of the invention. In summary, the package 100 includes a first housing for storing unused tissue papers, and a second housing for storing used tissue papers, wherein the first and second housing are attached to each other. A user retrieves unused tissue papers from the first housing, uses the tissue papers, and then disposes the used tissue papers into the second housing.

More specifically, the exemplary package 100 comprises a first housing 102 for storing unused tissue papers 150, and a second housing 104 for storing used tissue papers 160. The unused tissue papers 150 may be scented or unscented, and include anti-bacterial, moisturized and printed items.

In the exemplary embodiment, the second housing 104 is configured into a substantially cylindrical shape, with the diameter of the cylinder gradually increasing from the bottom to the top of the housing 104. This shape allows the second housing 104 to fit into a cup holder or other receptacle for use within a vehicle. The second housing includes a bottom 104a, a side wall 106, and an open top. The second housing 104 includes a removable lid 108 used to cover the top of the second housing 104. The lid 108 includes a circular top wall 108a and a ring-shaped side wall 108b extending downward from the perimeter of the circular wall 108a. When the lid 108 covers the second housing 104, the side wall 106b of the lid 108 extends downward along the exterior of the side wall 106 of the second housing 104. The lid 108 may be screwed on, push-on, or attached to (such as in a flip-up lid) the second housing 104.

Also, in the exemplary embodiment, the side wall 106 of the second housing 104 includes an opening 110 through which a user can insert used tissue papers 160 into the second housing 104. The opening 110 may be circular in shape. The second housing 104 may include a plurality of pie-shaped sub-covers 112, arranged together in a circular-fashion and their respective vertices coincident with each other, to substantially cover the opening 110. Adjacent sub-covers 112 are initially attached to each other by way of respective perforated lines.

With such configuration, when a user finishes using the first tissue paper, a user pushes the used tissue paper 160 through the plurality of sub-covers 112. The pushing force causes the sub-covers 112 to detach from each other and bend inward into the second housing 104, thereby allowing the user to insert the used tissue 160 into the second housing 104. After the user places the used tissue paper 160 into the second housing 104, the plurality of sub-covers 112 may have resilient characteristics causing them to bend back toward the opening 110 to at least further occlude the opening 110, thereby keeping the used tissue paper 160 essentially enclosed within the second housing 104. A liner bag (not shown) may be included within the second housing 104 to receive used tissue papers 160. The liner bag may be disposed upon being filled, and a new liner bag may be placed within the second housing 104. This allows the continued use of the second housing 104, which may be made out of plastic or other durable material. The interior of the second housing 104 and/or the bag may be coated with an anti-microbial substance for improved germicidal protection.

The first housing 102 is attached to the top side of the removable lid 108. The first housing 102 may be attached to the removable lid 108 by an adhesive, a two-sided adhesive tape, or by a mechanical device. The top side of the lid 108 may include a groove configured to receive and register with the bottom of the first housing 102. The first housing 102 includes an opening 102a through which a user can pull out the unused tissue papers. The first housing 102 may further include a cover (not shown) initially attached to the top side of the first housing 102 in a manner that substantially covers the opening 102a. The cover may be made out of a cardboard and attached to the top side of the first housing 102 by a perforated line. Alternatively, the cover may be made out of a transparent plastic. Before anyone uses of the tissue papers 150, a user needs to detach the detach or open the cover in order to access the unused tissue papers 150. The plurality of unused tissue papers 150 may be arranged in a vertically or horizontally stacked, folded, rolled or any other configuration within the first housing 102.

Although tissue papers are used to exemplify the various embodiments of the invention, it shall be understood that the various packages disclosed herein may store other disposable cleaning products, such as paper towels, napkins, and moist wipes.

While the invention has been described in connection with an exemplary embodiment, it will be understood that the invention is capable of further modifications. This application is intended to cover any variations, uses or adaptation of the invention following, in general, the principles of the invention, and including such departures from the present disclosure as come within the known and customary practice within the art to which the invention pertains.

What is claimed is:

1. A package comprising a first housing for storing unused, disposable cleaning products, and a second housing for storing used, disposable cleaning products, wherein said first housing is attached to said second housing.

2. The package of claim 1, wherein said first housing includes an opening through which said unused, disposable cleaning products are to be removed from said first housing.

3. The package of claim 2, further comprising said unused, disposable cleaning products situated within said first housing.

4. The package of claim 3, wherein said unused, disposable cleaning products are in a stacked arrangement within said first housing.

5. The package of claim 3, further comprising a cover attached to said first housing in a manner that substantially covers said opening.

6. The package of claim 1, wherein said second housing includes an opening through which said used disposable cleaning products are to be inserted into said second housing.

7. The package of claim 6, further comprising a cover to at least partially occlude said opening.

8. The package of claim 7, wherein said cover comprises a plurality of pie-shaped sub-covers with vertices substantially coincident, wherein adjacent sub-covers are initially attached to each other by way of respective perforated lines.

9. The package of claim 1, further comprising a bag situated within said second housing.

10. The package of claim 9, wherein said bag includes an anti-microbial substance.

11. The package of claim 1, wherein said second housing includes a lid.

12. The package of claim 11, wherein said first housing is attached to said second housing by way of said lid.

13. The package of claim 12, wherein said lid includes a groove configured to receive and register with said first housing.

14. The package of claim 11, wherein said lid is removable.

15. The package of claim 14, wherein said lid is removable by a screwing action, pulling action, or hinging action.

16. The package of claim 1, wherein said unused disposable cleaning products comprises any one of the following:
tissue paper;
paper towels;
napkins; or
moist wipes.

17. The package of claim 1, wherein said second housing is configured into a cylinder.

18. The package of claim 17, wherein a diameter of said cylinder increases gradually from a bottom of said housing to a top of said housing.

19. A method, comprising:
removing an unused, disposable cleaning product from a first housing that is attached to a second housing;
using said disposable cleaning product; and
inserting said used, disposable cleaning product into said second housing.

20. The method of claim 19, wherein said disposable cleaning product comprises any one of the following:
a tissue paper;
a paper towel;
a napkin; or
a moist wipe.
